(19) United States
(12) Patent Application Publication
(10) Pub. No.: US 2016/0029921 A1
(43) Pub. Date: Feb. 4, 2016

(54) LEAK DETECTION

(71) Applicant: EMPIRE TECHNOLOGY DEVELOPMENT LLC, Wilmington, DE (US)

(72) Inventor: Debasish PRADHAN, Sambalpur (IN)

(21) Appl. No.: 14/782,787

(22) PCT Filed: Feb. 28, 2014
(86) PCT No.: PCT/US2014/019653

§ 371 Date: Oct. 6, 2015

(30) Foreign Application Priority Data
Apr. 6, 2013 (IN) 1030/DEL/2013

(51) Int. Cl. A61B 5/07; A61B 5/1473; A61B 5/00

(52) U.S. Cl. CPC A61B 5/076; A61B 5/4255; A61B 5/1473; A61B 5/746; A61B 5/6873; A61B 2562/0204; A61B 2562/18; A61B 2560/0214

(57) ABSTRACT

Some embodiments described herein generally relate to apparatus and methods for detecting gases in a body of a human or other animal. For example, such gases may be those normally contained within an organ of the body. The detected gases may, thus, indicate leakage of the contents of the organ into a body cavity. The apparatus may include one or more gas sensing probes at least partially positioned within a semipermeable material and configured to detect an intestinal gas and an electronic circuit coupled to the gas sensing probes. The electronic circuit may include a transmitter configured to transmit information from the gas sensing probes to an external device.

### BACKGROUND

Unless otherwise indicated herein, the materials described herein are not prior art to the claims in the present application and are not admitted to be prior art by inclusion in this section.

Anastomosis is a surgical procedure in which two structures are connected to one another inside the body. Anastomosis is commonly used in surgical resection of a portion of the colon, a procedure referred to as a colectomy. For example, after the portion of the colon is removed, remaining upper and lower sections of the colon may be attached to one another to restore the bowel. Anastomosis may also be used in surgical procedures on the small intestine, stomach, esophagus, bladder and/or bile duct. For example, in gastric bypass surgery, a portion of the small intestine is resected and rerouted to a small stomach pouch.

Hundreds of thousands of anastomoses are performed each year. Unfortunately, postoperative complications occurring as a result of the anastomosis are not uncommon. For example, complications such as abscesses, mechanical obstructions and anastomotic leaks may be caused by defects in or breakdown along the site of the anastomosis. Colorectal anastomotic leakage is a major complication of colorectal anastomosis in which a leak develops in the areas where two parts of the gastrointestinal tract are reattached. As fluids, such as gastrointestinal content, leak through the site of the anastomosis out of the colon, the normally sterile peritoneal cavity becomes contaminated. The anastomotic leak may lead to sepsis or peritonitis, which is an infection of the peritoneal cavity that may be lethal. The chance of an anastomotic leak forming after surgery is up to 15% and is more common in patients whose wounds may take longer to heal, such as the elderly, diabetics and patients suffering from cancer. Mortality following anastomotic leakage can range from 6% to 40%. Anastomotic leakage is particularly dangerous because it is linked with an increased recurrence of malignancy, which has been attributed to formation of tumor cells through the anastomotic site. Additionally, formation of abscess and fistula due to dehiscence are often difficult to treat. Nearly half of all patients with anastomotic leakage require surgical intervention, such as fecal diversion, with the other half requiring antibiotics and drainage.

Current methods of diagnosing a potentially life-threatening anastomotic leak include monitoring clinical symptoms and blood testing. Most frequently, the clinical symptoms, such as fever, acute abdominal pain, breathing difficulties and neurologic irregularities, do not present in a patient until the patient has developed an infection, inflammatory syndrome, or even sepsis, which may occur weeks after surgery. Similarly, the blood testing performed to diagnose an anastomotic leak detects the presence of white blood cells indicating an infection in the body that develops long after the initial anastomotic leak forms. Thus, such methods are not capable of detecting the presence of the anastomotic leak before the patient has developed symptoms or sepsis.

### SUMMARY

The technologies described herein generally relate to apparatus and methods for detecting leakage from bodily organs.

In some examples, an apparatus for detecting leakage from an organ is disclosed. The apparatus may include at least one gas sensing probe at least partially positioned within a semipermeable material and configured to detect an intestinal gas and an electronic circuit coupled to the at least one gas sensing probe and including a transmitter configured to transmit information from the at least one sensor to an external device.

In some examples, a system for detecting leakage from a colon is disclosed. For example, the system may include a probe including at least one gas sensor configured to detect at least one of methane gas and hydrogen sulfide gas, an electronic circuit coupled to the at least one gas sensor and including an electronic transmitter and an external receptor configured to receive a signal from the electronic transmitter the electronic circuit. The probe may be sized and configured to circumscribe at least a portion of the colon.

In some examples, a method for detecting leakage from an organ is disclosed that includes introducing an apparatus into a region of a body proximate an organ, the apparatus including an electronic circuit coupled to a probe including at least one gas sensor encapsulated within a semipermeable material, positioning the electronic circuit within a wall of the body such that at least a portion of the probe protrudes into a cavity of the body containing the organ and monitoring the at least one gas sensor to determine the presence of gases leaked from the organ.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

In the drawings:

FIG. 1 illustrates an embodiment of an apparatus for detecting gases in a body cavity;

FIG. 2 illustrates an enlarged, cross-sectional side view of an embodiment of a section of an apparatus such as that of FIG. 1;

FIG. 3 illustrates an enlarged, cross-sectional side view of an embodiment of an end section of an apparatus such as that of FIGS. 1 and 2;

FIGS. 4A and 4B illustrate an embodiment of a sheath that may be used in the apparatus of FIGS. 1 and 2;

FIG. 5 illustrates another embodiment of an apparatus for detecting gases in a body cavity;

FIG. 6 illustrates another embodiment of an apparatus for detecting gases in a body cavity;

FIG. 7 illustrates another embodiment of an apparatus for detecting gases in a body cavity;

FIGS. 8A through 10B illustrate a top down view and a side view of additional embodiments of apparatus for detecting gases in a body cavity;

FIG. 11 shows an example flow diagram of a method for detecting leakage from a colon using the apparatus of FIGS. 1 through 6; and

all arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Some embodiments described herein generally relate to apparatus and methods for detecting gases in a body of a human or other animal. For example, such gases may be those normally contained within an organ of the body. The detected gases may, thus, indicate leakage of the contents of the organ into a body cavity. As used herein, the terms “body cavity” and “cavity” may refer to any space in a body of a human or animal, which may be fluid-filled. For example, a body cavity may be a region positioned within a membrane where internal organs develop. Examples of such body cavities include the peritoneal cavity, the thoracic cavity, and the pelvic cavity. As a non-limiting example, detection of intestinal gases, such as methane gas and hydrogen sulfide gas, within the peritoneal cavity may indicate the presence of an anastomotic leak and/or an infection. The apparatus may be placed in close proximity to a closed incision, such as an anastomosis, or a lesion within an organ. As used herein, the terms “anastomotic” and “anastomosis” may refer to a joining or union of organs or parts that are normally separate within a body of a human or animal. For example, an anastomosis may include a surgical connection of severed tubular organs or parts to form a continuous channel, such as between two parts of the intestine of a human or animal.

The apparatus may include a probe including one or more gas sensors positioned within a sheath and an electronic circuit operably coupled to the probe. The gas sensors may be, for example, targeted gas sensors configured to detect one or more of gases. As a non-limiting example, each of the sensors may include at least one material reactive with the gas. In some embodiments, the sensors may include a metal-oxide-semiconductor device, a conducting polymer, a quartz crystal resonator, or a surface acoustic wave device. The sheath may be formed from a semipermeable material including an internal passageway and the sensors within the sheath may be surrounded by filter material, such as an activated carbon material. The electronic circuit may be part of a flexible electronic chip and may be configured to transmit a signal indicating detection of the gases by the sensors. By way of example and not limitation, the electronic chip may have dimensions of less than about 10 mm.

Such apparatus may be useful in detecting the presence of an anastomotic leak from an intestinal organ, such as a colon. The probe may be positioned in a region of the peritoneal cavity proximate the anastomosis formed to reconnect portions of the colon. The electronic circuit coupled to the gas sensors of the probe may be positioned in a region of a body wall surrounding the peritoneal cavity. In some embodiments, the probe and the electronic chip may be inserted through a laparoscopic opening used to perform the anastomosis. The gas sensors may detect gases arising from or seeping out of the anastomotic leak and may transmit signals via the electronic chip to enable accurate and precise detection of leakage. For example, the electronic chip may transmit an electromagnetic signal or a wireless signal to an external device, such as a mobile phone.

The apparatus and methods described herein provide immediate and localized detection of leaks from bodily organs, such as anastomotic leaks. The apparatus and methods described herein provide a convenient form of detecting anastomotic leakage before clinical symptoms arise and, thus, may advantageously prevent infections and complications associated with such leakage.

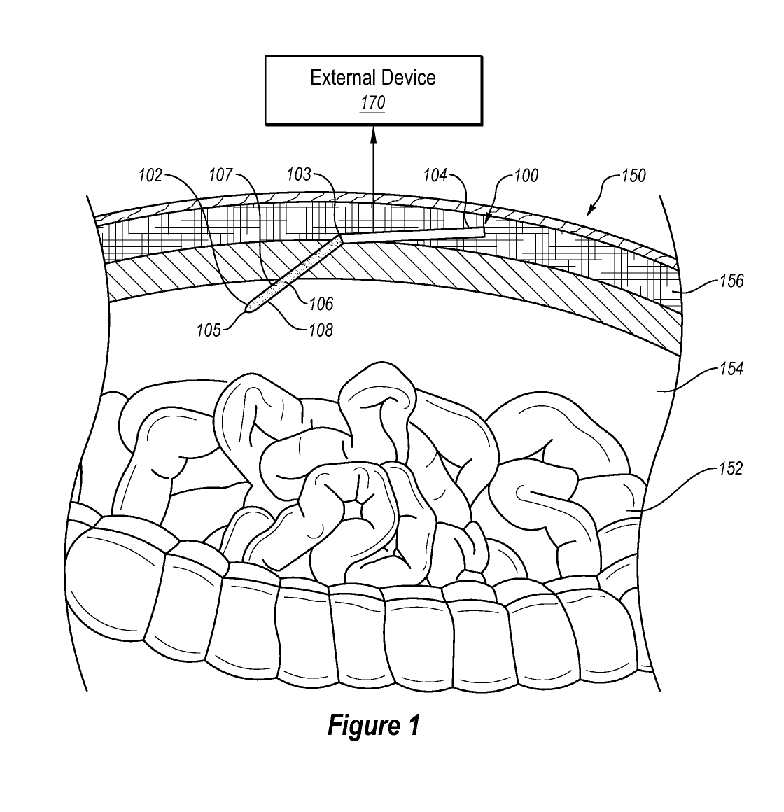

FIG. 1 illustrates an embodiment of an apparatus for detecting gases in a body cavity, arranged in accordance with at least some embodiments described herein. The apparatus 100 may be configured to detect gases leaking from one or more organs, such as those of a gastrointestinal tract 152. The apparatus 100 may be used, for example, to determine the presence of an anastomotic leak by detecting intestinal gases produced by the organ of the gastrointestinal tract 152, such as a colon. While the apparatus 100 is described as being used to detect the intestinal gases resulting from the anastomotic leak in the gastrointestinal tract 152, it will be understood that the apparatus 100 may be used to detect leakage from other bodily organs.

The apparatus 100 may include a probe 102 having a proximal end 103 coupled to an electronic chip 104 and a distal end 105 opposite the proximal end 103. The distal end 105 may be configured to be introduced into a cavity 154 of the body 150 positioned between the gastrointestinal tract 152 and a body wall 156. For example, the body cavity 154 of the body 150 may be a peritoneal cavity. The probe 102 may include one or more sensors 106, each of which is configured to detect the presence of one or more gases. The sensors 106 may be positioned within a passageway 107 of a sheath 108, which may be formed from a rigid or semi-rigid material that is permeable to the gases. The passageway 107 may be a hollow, interior space within the sheath 108.

By way of example and not limitation, the sensors 106 may be positioned in the sheath 108 such that the probe 102 may be used in detecting different gases within the body cavity 154 of the body 150. For example, the sensors 106 may interact with or react to volatile compounds on contact resulting in a physical or electrical change in the sensors 106. In embodiments in which the apparatus 100 is used to detect the anastomotic leak, the probe 102 may include one or more of the sensors 106 configured to detect methane (CH₄) gas and one or more of the sensors 106 configured to detect hydrogen sulfide (H₂S) gas.

For example, the sensors 106 may form a so-called “electronic nose” that detects and measures odorant and non-odorant volatile compounds. The sensors 106 may be formed in a variety of miniaturized sizes using, for example, thin-film semiconductors, micro- and nano-electronics and/or highly sensitive, low power-based gas sensors. By way of example and not limitation, the sensors 106 may be formed from one or more of a metal oxide-based sensor, a nanowire-based sensor, a conducting polymer-based sensor, a conducting polymer-metal nanoparticle hybrid-based sensor, a quartz crystal resonator, and a surface acoustic wave device. The metal oxide-based sensor may be a metal-oxide metal-oxide-semiconductor device configured to generate a change in electrical resistance when the gases are adsorbed onto a surface thereof. The change in electrical resistance may vary depending on a type and/or concentration of the gases. The nanowire-based sensor may include a single nanowire and metal oxide nanoclusters chosen to react to the gases. The conducting polymer-metal nanoparticle hybrid-based sensor may include metal nanoparticles formed on functionalized conducting polymer film surfaces with conjugated linker molecules.

The probe 102 may be coupled to the electronic chip 104 by electrical interconnects or wires, for example. The electronic chip 104 may be embedded within the body wall 156 of the body 150. By way of example and not limitation, the electronic chip 104 may have dimensions of less than about 10 mm and, more particularly, between about 0.5 mm and about 7 mm and, yet more particularly, between about 3 mm and about 5 mm, such that it may be readily inserted into a wall of the body. Such dimensions of the electronic chip 104 are provided as examples and it will be understood that the electronic chip 104 may be formed having any suitable dimensions.

The electronic chip 104 may include, for example, an electronic circuit formed on a flexible substrate. As a non-limiting example, the electronic chip 104 may be a flexible, stretchable semiconductor element that includes the electronic circuit formed on a thin layer of semiconductor material that is bonded to a flexible supporting substrate. As another non-limiting example, the electronic chip 104 may be a flexible wireless monitoring system formed using a three-dimensional integration process. By way of example and not limitation, a semiconductor chip having a thickness of between about 10 μm and about 50 μm and, more particularly between about 20 μm and about 30 μm, may be embedded in a flexible ultra-thin chip package or a flexible printed circuit board (PCB) such that the electronic chip 104 has a thickness of between about 40 μm and about 100 μm and, more particularly, between about 60 μm and about 80 μm. Such thicknesses of the semiconductor chip and the electronic chip 104 are provided as examples and it will be understood that the semiconductor chip and the electronic chip 104 may be formed having any suitable thickness.

For example, the electronic circuit may include a wireless transmitter configured to transmit a wireless signal to an external device 170. The external device 170 may be an electronic device or mobile device, such as a computer, a mobile phone, a smart phone, a tablet computer, a personal digital assistant (PDA), and the like.

The apparatus 100 may be introduced into the body 150, for example, through the opening in the body wall 156. The apparatus 100 may be positioned such that the probe 102 at least partially extends into the body cavity 154 and the electronic chip 104 is positioned within the body wall 156. The electronic chip 104 may optionally be secured to the body wall 156 using a stitch, suture or clip, for example. The probe 102 may be positioned proximate to the anastomosis in the organ of the gastrointestinal tract 152, such as the colon. By way of example and not limitation, the apparatus 100 may be introduced into the body cavity 154 through a laparoscopic opening in the body wall 156 of the body 150, which may have a size of between about 3 mm and about 20 mm, and more particularly, between about 5 mm and about 18 mm. The sensors 106 may detect the presence of specific gases within the body cavity 154 and the electronic chip 104 may transmit a signal to the external device 170 indicating the detection of the gases. Additionally, the electronic chip 104 may be configured to utilize heat from the body 150 as a power source. For example, the electronic chip 104 may include a thermoelectric generator to convert heat collected from the body 150 into electrical energy.

The external device 170 may receive the signal from the transmitter of the electronic chip 104 and may generate an electronic notification or message to notify a user that the gases were detected in the body 150. For example, the signal received by the external device 170 may indicate that the intestinal gases were detected in the body cavity 154, indicating the presence of the anastomotic leak. The external device 170 may be a portable electronic device worn or carried by a user, such as a patient or a healthcare professional. The external device 170 may be configured to generate the electronic notification, such as an alarm, or an electronic message for display on the external device 170 to notify the user of the detection of the gases.

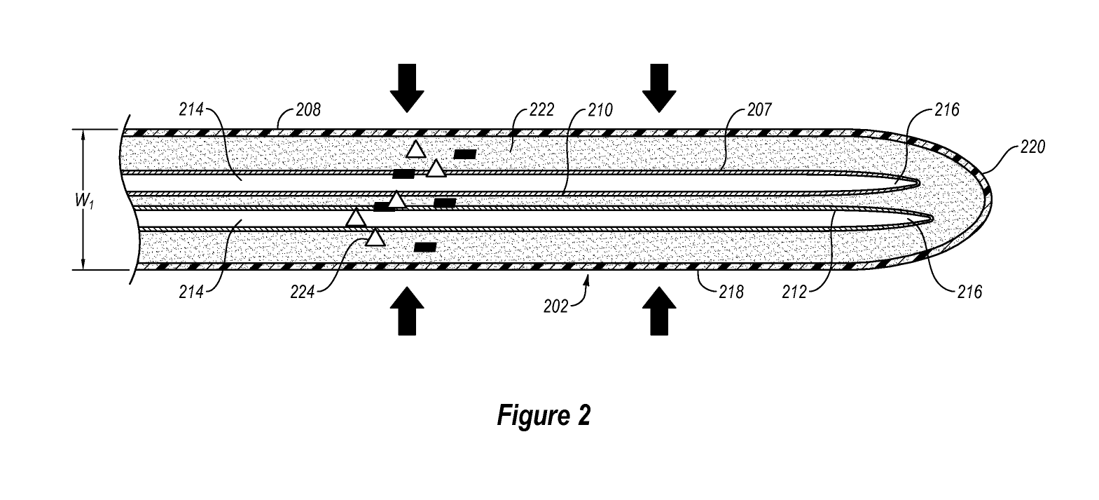

FIG. 2 illustrates an enlarged, cross-sectional side view of an embodiment of a section of an apparatus such as that of FIG. 1, arranged in accordance with at least some embodiments described herein. As shown in FIG. 2, probe 202 may include first and second gas sensing probes 210 and 212 positioned within a sheath 208. For the sake of simplicity, the apparatus 200 shown in FIG. 2 is illustrated as including two gas sensing probes 210 and 212. However, it will be understood that the apparatus 200 may include any number of such gas sensing probes.

The gas sensing probes 210 and 212 include sensors configured to detect the presence of the gas, such as the sensors 106 of FIG. 1. The sensors included in each of the gas sensing probes 210 and 212 may be configured to detect the same gas, or different gases. In embodiments in which the probe 202 is used in the detection of an anastomotic leak, the first gas sensing probe 210 may include methane gas sensors and the second gas sensing probe 212 may include hydrogen sulfide sensors. The gas sensing probes 210 and 212 may each include an elongated body 214 and a distal end 216 having a tapered or beveled configuration. The shape of the gas sensing probes 210 and 212 shown in FIG. 2 is provided as an illustrative example, and it will be understood that the gas sensing probes 210 and 212 may have any suitable configuration.

The sheath 208 may be configured to house the gas sensing probes 210 and 212. In embodiments in which the gas sensing probes 210 and 212 include the elongated body 214 and the tapered distal end 216, the sheath 208 may be formed having an elongated body 218 and a tapered distal end 220. The sheath 208 may be formed from a material that is rigid or semi-rigid. Additionally, the material used to form the sheath 208 may be formed from a material that is semi-permeable or permeable to one or more of the gases. As used herein, the term “permeable” may refer to a material through which one or more gases may pass, and encompasses the term “semipermeable.” As used herein, the term “semipermeable” may refer to a material through which certain molecules or ions may pass while other, larger molecules or ions cannot pass. For example, the sheath 208 may be formed from a permeable or semipermeable, biocompatible, polymeric material, such as polytetrafluoroethylene (PTFE), polyurethane (PU), polyethylene (PE), nylon, and the like.

By way of example and not limitation, the pore size of the material used to form the sheath 208 may be between about 0.5 μm and about 10 μm, and more particularly, about 2 μm and about 4 μm. The sheath 208 may be formed from membrane having a pore size that filters out solids, liquids and/or gases with higher molecular size. In embodiments in which the apparatus is used to detect an anastomotic leak, the sheath 208 may be formed from a material that only allows methane and hydrogen sulfide gases to pass through the sheath 208.

For example, the probe 202 may have a width W₁ of less than about 10 mm and, more particularly, between about 2 mm and about 8 mm. The width W₁ of the probe 202 may be determined based on a type of surgical procedure (e.g., a minimally invasive procedure or an open surgical procedure), a size of a device used to deploy the apparatus 200, anatomy of the body and/or proximity of the probe 202 to a detection site (e.g., the anastomosis).

A filter 222 formed from an absorbent or adsorbent material may be positioned in the sheath 208 surrounding the gas sensing probes 210 and 212. The filter 222 may be formed from a material that adsorbs or absorbs the gases that the sensors are configured to detect. For example, in embodiments in which the sensors are configured to detect methane and/or hydrogen sulfide gas to determine the presence of the anastomotic leak, the filter 222 may be configured to adsorb or absorb the methane and/or hydrogen sulfide gas. The filter 222 may be a chemical or carbon-compound filter, such as an activated carbon filter. The activated carbon filter may be impregnated with alkaline or other chemicals to impart the filter with an increased adsorption capacity for hydrogen sulfide gas. The filter 222 may also include boron nitride nanotubes (BNNTs) or carbon nanotubes (CNTs) to impart the filter with an increased adsorption capacity for methane gas. By way of example and not limitation, the filter 222 may include a combination of activated carbon, carbon nanotubes and/or boron nitride nanotubes.

Molecules 224 of the gases may enter the probe 202 through the sheath 208, as illustrated by directional arrows. The gases may flow through the filter 222 and into the passageway 207 surrounding each of the gas sensing probes 210 and 212.

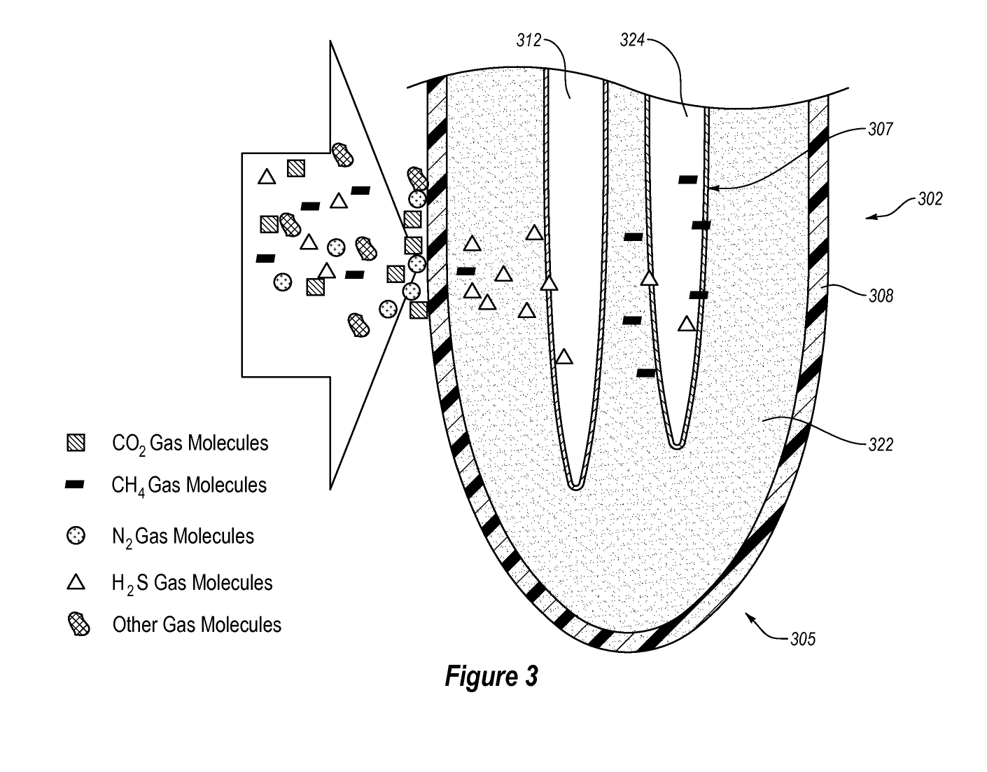

FIG. 3 illustrates an enlarged, cross-sectional side view of an embodiment of an end section of an apparatus such as that shown in FIGS. 1 and 2, arranged in accordance with at least some embodiments described herein. The sheath 308 is formed from a material permeable to methane (CH₄) and hydrogen sulfide (H₂S) and impermeable to carbon dioxide (CO₂) gas, nitrogen (N₂) gas and other gas molecules. The probe 302 may be positioned in the peritoneal cavity proximate the site of the anastomosis in the colon, for example. The anastomotic leak may occur at the site of the anastomosis in the colon resulting in leakage of gases from the colon into the peritoneal cavity. Such leaked gases may include, for example, methane gas and hydrogen sulfide gas.

The molecules 324 of various gases, including the methane gas, the hydrogen sulfide gas, carbon dioxide gas, nitrogen gas and other gases, may be present in a body cavity surrounding the probe 302. The methane gas molecules and hydrogen sulfide gas molecules may pass through the sheath 308 and into the passageway 307 of the probe 302. For example, the sheath 308 may be selectively permeable to the methane gas molecules and hydrogen sulfide gas molecules and may be impermeable to one or more of the carbon dioxide gas molecules, the nitrogen gas molecules and other gas molecules. The filter 322 may absorb or adsorb the methane gas and/or the hydrogen sulfide gas to assist in detection by drawing the methane gas and/or the hydrogen sulfide gas to the gas sensing probes 310 and 312. In the passageway 307 of the probe 302, the molecules of the methane and hydrogen sulfide gases may come into contact with the sensors of the gas sensing probes 310 and 312. The sensors within the gas sensing probes 310 and 312 may detect the methane and hydrogen sulfide gases and may generate a response signal that may be transmitted by an electronic chip to an external device, such as the electronic chip 104 and the external device 170 described with respect to FIG. 1.

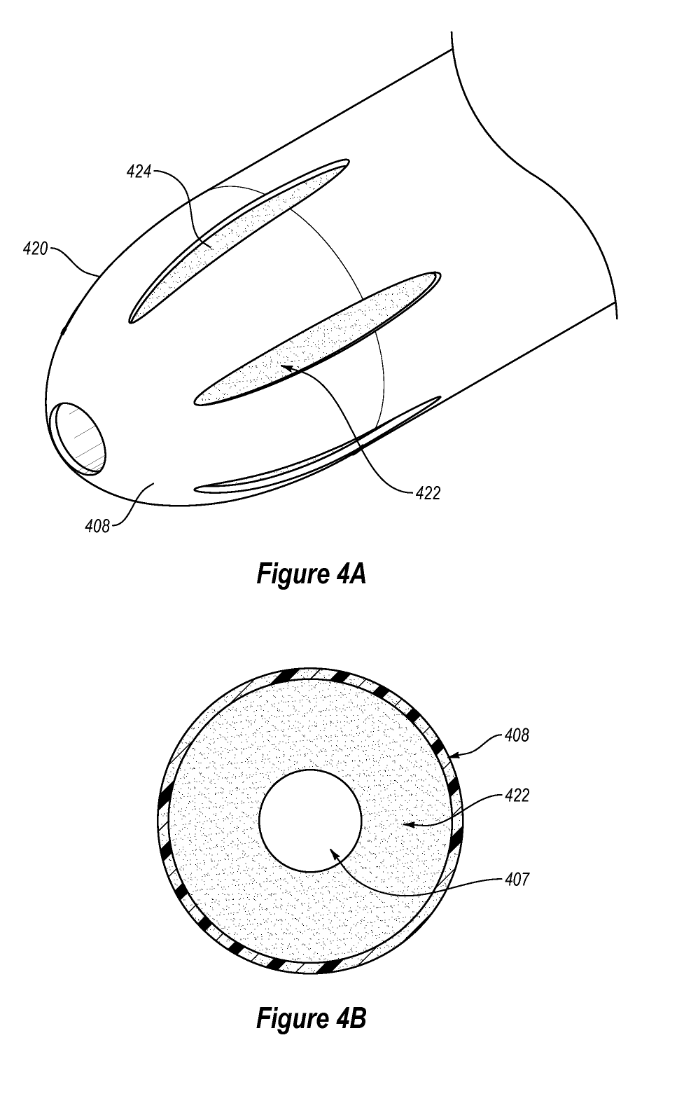

FIGS. 4A and 4B illustrate an embodiment of a sheath that may be used in the apparatus of FIGS. 1 and 2, arranged in accordance with at least some embodiments described herein. A sheath 408 configured to house one or more of the sensors or the gas sensing probes may include one or more apertures 424 that facilitate the flow of gases into a passageway 407. The sheath 408 may be formed from substantially the same materials as the sheath 108 described with respect to FIGS. 1 through 3. Additionally, the material used to form the sheath 408 may be substantially impermeable to fluids and gases such that the gases enter the sheath 408 only through the apertures 424. The passageway 407 may be substantially surrounded by a filter 422, which may be formed from substantially the same materials as the filter 122 described with respect to FIGS. 1 through 3. As a non-limiting example, the apertures 424 in the sheath 408 may be positioned in a predetermined pattern such as a circumferential array. For example, at least a portion of the apertures 424 may be positioned in substantial alignment around a circumference of the sheath 408. For the sake of simplicity, the apertures 424 shown in FIG. 4A are positioned near a distal end 420 of the sheath 408. However, it will be understood that the sheath 408 may include any number of such apertures 424 along a length of the sheath 408. The apertures 424 may be generally spaced an equidistance apart, or may be positioned randomly.

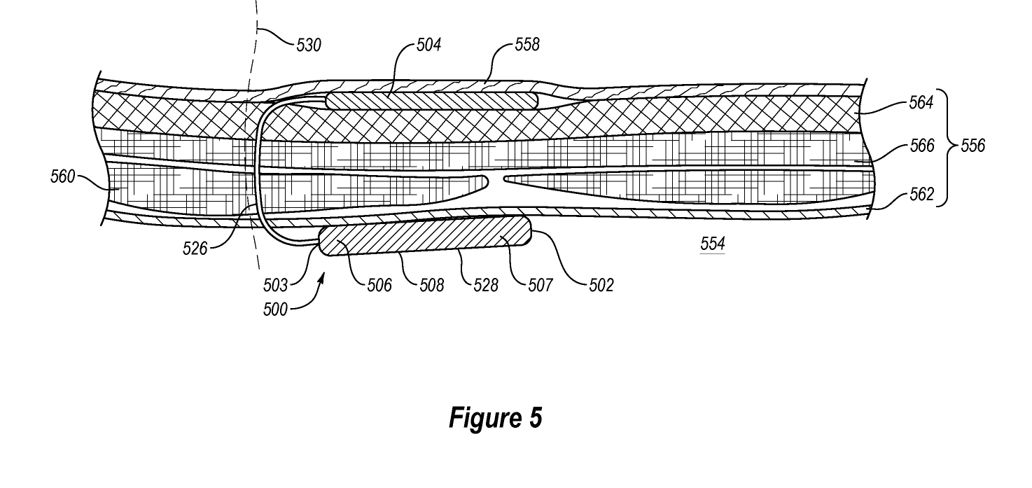

FIG. 5 illustrates another embodiment of an apparatus for detecting gases in a body cavity, arranged in accordance with at least some embodiments described herein. The apparatus 500 may include a probe 502 and an electronic chip 504 respectively having substantially the same configuration as the probe 102 and the electronic chip 104 described with respect to FIG. 1. The apparatus 500 may be introduced into the body, for example, through an opening in the body wall 556. For example, the opening may be formed in the body wall 556 along incision line 530. The probe 502 may be electrically coupled to the electronic chip 504 by an electrical interconnect 526, such as a cable or wire, for example.

The electronic chip 504 may be positioned within the body wall 556 and the probe 502 may be positioned at least partially within the body cavity 554 such that a surface 528 of the probe 502 is exposed within the body cavity 554. For example, the electronic chip 504 may be implanted or inserted in a dermis 558 of the body wall 556, or between an abdominal layer 560 and a peritoneum 562 underlying a fascia layer 564 and a fat layer 566. The probe 502 may include a sheath 508 having an internal lumen 507 in which gas sensors 506 are positioned. The sheath 508 may include one or more permeable or semipermeable regions. The surface 528 of the probe 502 exposed within the body cavity 554 may include the permeable or semipermeable region of the sheath 508 of the probe 502. The apparatus 500 may be positioned within the body such that the surface 528 is positioned adjacent an incision in an organ, such as an anastomosis in a colon.

The electrical interconnects 526 may extend from a proximal end 503 of the probe 502, through the body wall 556, to the electronic chip 504. As a non-limiting example, the electrical interconnects 526 may function as a fastener or clip that clamps the intervening portions of the body wall 556 between the probe 502 and the electronic chip 504.

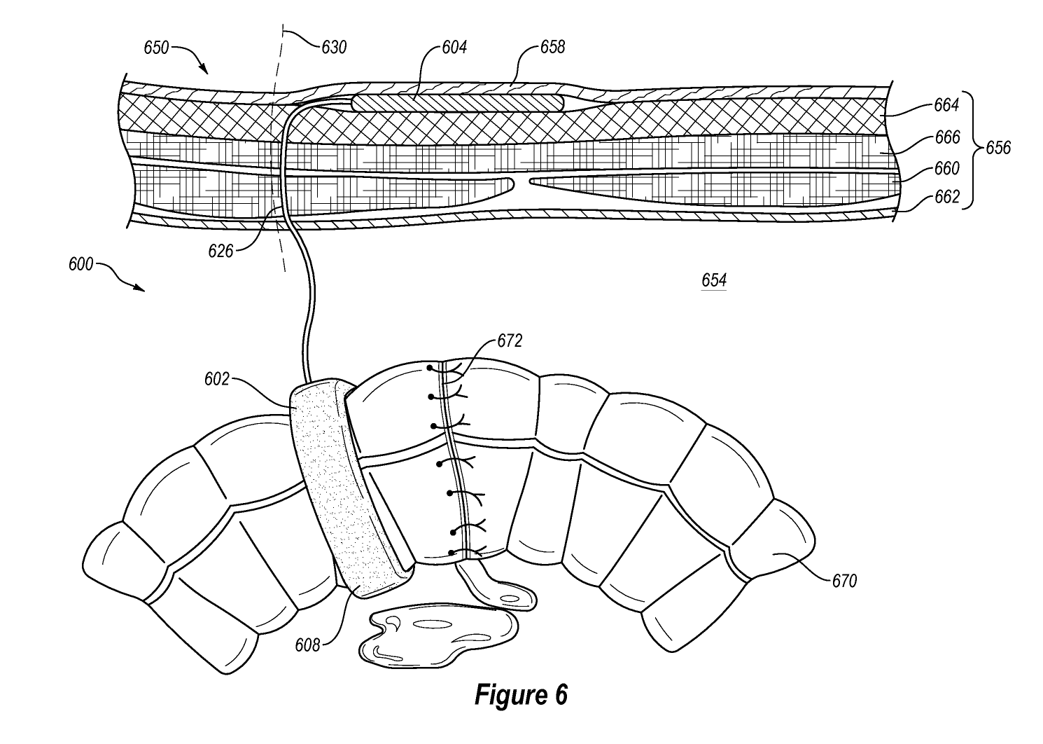

FIG. 6 illustrates another embodiment of an apparatus for detecting gases in a body cavity, arranged in accordance with at least some embodiments described herein. The apparatus 600 may include a ring-shaped probe 602 coupled to an electronic chip 604 by electrical interconnect 626. The ring-shaped probe 602 may include one or more gas sensors (not shown), such as the gas sensors 106 described with respect to FIG. 1. The gas sensors may be positioned with a ring-shaped sheath 608, which may be formed from substantially the same materials as the sheath 108 described with respect to FIG. 1. The apparatus 600 may be introduced into the body, for example, through an opening (not shown) in the body wall 656. The electronic chip 604 may be implanted or inserted in a dermis 658 of the body wall 656, or between an abdominal layer 660 and a peritoneum 662 underlying a fascia layer 664 and a fat layer 666. For example, the opening may be formed in the body wall 656 along incision line 630. While the apparatus shown in FIG. 6 is positioned around a circumference of a gastrointestinal organ 670, it is to be understood that the probe 602 may be positioned around any organ of a human or animal.

As a non-limiting example, the ring-shaped probe 602 may be sized and configured to be positioned around at least one portion of the gastrointestinal organ 670, such as the small intestine, the colon, the stomach(s), the esophagus, etc. The ring-shaped probe 602 may be positioned proximate or overlying an anastomosis 672 in the gastrointestinal organ 670, for example. As a non-limiting example, the ring-shaped probe 602 may have a length sufficient to circumscribe at least a portion of a human colon. In this configuration, the ring-shaped probe 602 may have a length of between about 5 millimeters (mm) and about 60 mm and, more particularly, between about 10 mm and about 40 mm. The circumferential length of the ring-shaped probe 602 may be increased or decreased depending, for example, on the size of the organ or the body cavity 654 within a particular human or animal in which the apparatus 600 will be used to detect the presence of gases.

The electronic chip 604 may be positioned within the body wall 656. For example, the electronic chip 604 may be implanted or inserted in the dermis 658, or between the abdominal layer 660 and the peritoneum 662. The electrical interconnects 626 may extend from the ring-shaped probe 602, through a portion of the body wall 656, to the electronic chip 604. The electronic chip 604 may be configured to receive signals from the probe 602 via the electrical interconnects 626. The signals may indicate detection of gases by the probe 602 and to transmit the signal to an external device (not shown), such as the external device 170 described with respect to FIG. 1.

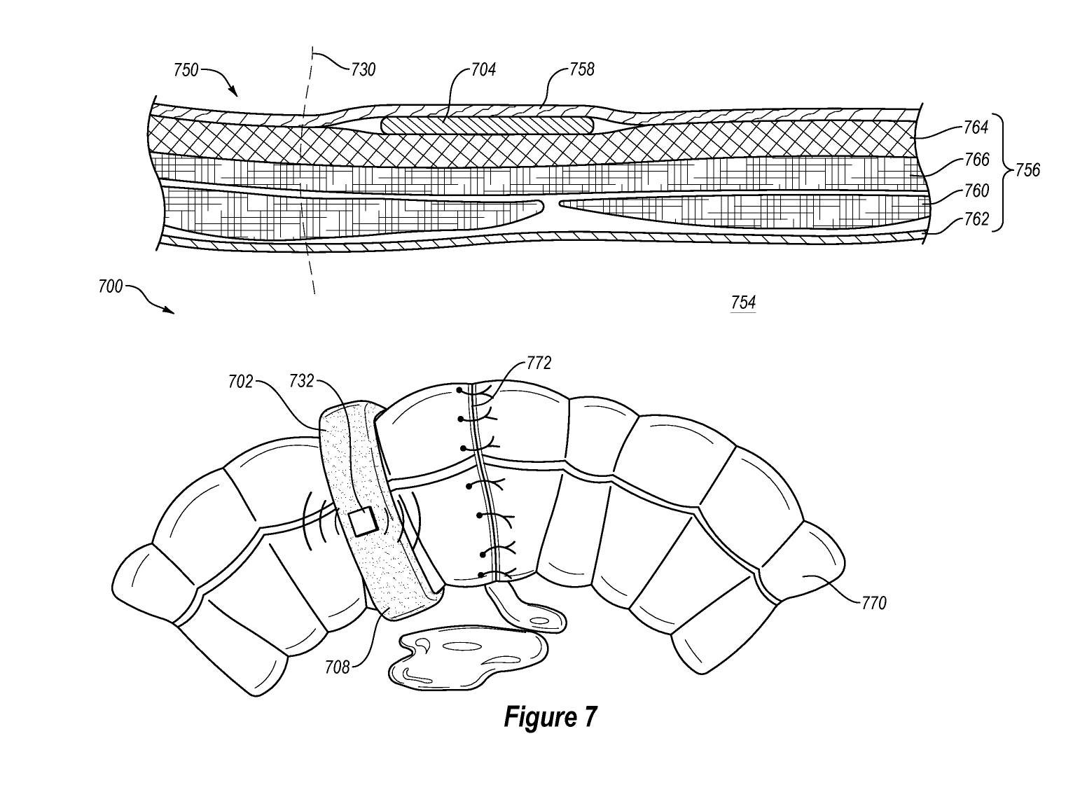

FIG. 7 illustrates another embodiment of an apparatus for detecting gases in a body cavity, arranged in accordance with at least some embodiments described herein. The apparatus 700 may include a ring-shaped probe 702 having one or more gas sensors, such as the gas sensors 106 described with respect to FIG. 1, positioned within a ring-shaped sheath 708. The ring-shaped probe 702 may be sized and configured to at least partially circumscribe a region of the gastrointestinal organ 770 near the anastomosis 772. The ring-shaped probe 702 of the apparatus 700 may include a wireless transmitter 732 configured to transmit a wireless signal to an electronic chip 704. The electronic chip 704 may include an integrated receptor configured to receive signals from the probe 702 via the wireless transmitter 732. The signals may indicate detection of gases by the probe 702 which may trigger the wireless transmitter 732 to transmit the signal to an external device (not shown), such as the external device 170 described with respect to FIG. 1.

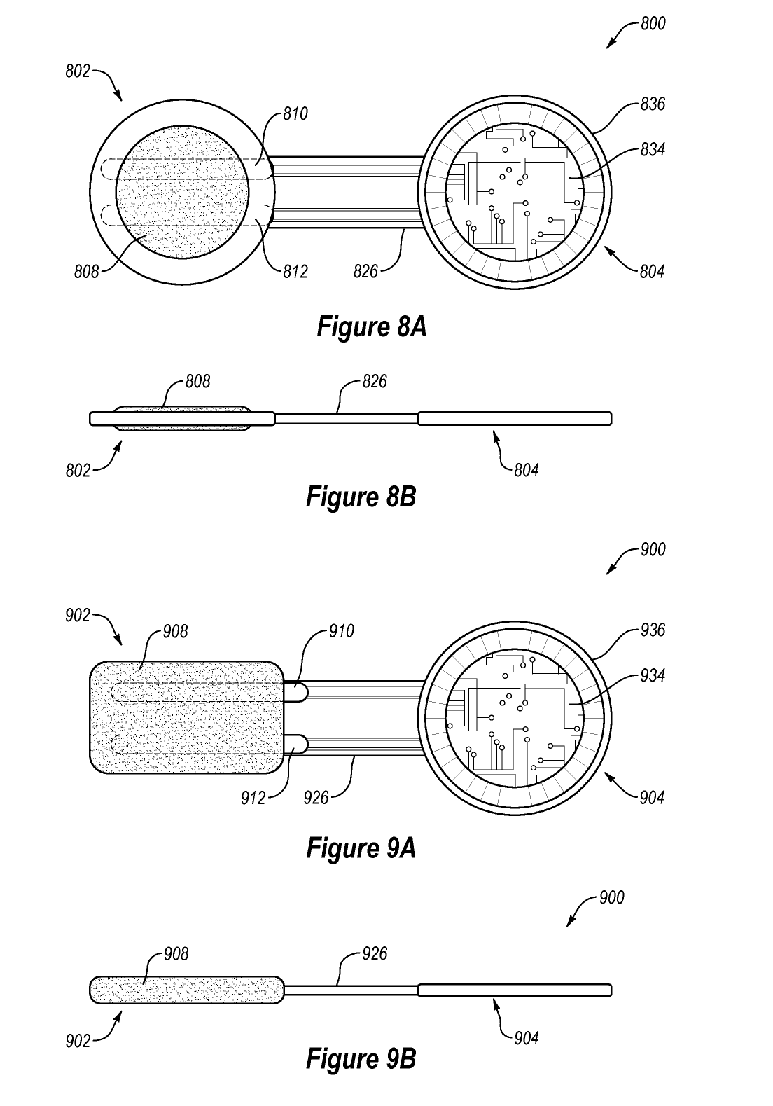

FIGS. 8A through 10B illustrate a top down view and a side view of additional embodiments of apparatus for detecting gases in a body cavity, each arranged in accordance with at least some embodiments described herein. Each of the apparatus described with respect to FIGS. 8A through 10B includes a probe including gas sensors and a flexible electronic chip coupled to the probe.

FIGS. 8A and 8B illustrate another embodiment of an apparatus 800 for detecting gases in a body cavity, arranged in accordance with at least some embodiments described herein. FIG. 8A illustrates a top down view of the apparatus 800 and FIG. 8B illustrates a side view of the apparatus 800. The apparatus 800 may include a probe 802 including gas sensing probes 810 and 812 having a rod-shape positioned within a sheath 808 having a circular, disc-shape. The gas sensing probes 810 and 812 may include one or more sensors configured to detect the presence of a gas, and may be coupled to an electronic chip 804. For example, the gas sensing probes 810 and 812 may be substantially the same as the gas sensing probes 210, 212, 310 and 312 described with respect to FIGS. 2 and 3. The sheath 808 may be formed from a biocompatible, semipermeable or permeable, rigid or semi-rigid material as described with respect to the sheath 108 of FIG. 1.

The electronic chip 804 may also have a circular, disc shape, as shown herein, or any other suitable shape. The electronic chip 804 may include an electronic circuit 834 having substantially the same configuration as the electronic chip 104 described with respect to FIG. 1. For example, the electronic circuit 834 of the electronic chip 804 may be formed on a flexible substrate 836. The electronic chip 804 may be coupled to the gas sensing probes 810 and 812 by electrical interconnects 826. The electronic circuit 834 may include a wireless transmitter configured to transmit a wireless signal indicating detection of the gas by the gas sensing probes 810 and 812.

FIGS. 9A and 9B illustrate another embodiment of an apparatus 900 for detecting gases in a body cavity, arranged in accordance with at least some embodiments described herein. FIG. 9A illustrates a top down view of the apparatus 900 and FIG. 9B illustrates a side view of the apparatus 900. The apparatus 900 may include a probe 902 including gas sensing probes 910 and 912 having a rod-shape positioned within a sheath 908 having a rectangular shape. The sheath 908 may be formed from a biocompatible, semipermeable or permeable, rigid or semi-rigid material as described with respect to the sheath 108 of FIG. 1. The gas sensing probes 910 and 912 may include one or more sensors configured to detect the presence of a gas, and may be coupled to an electronic chip 904. For example, the gas sensing probes 910 and 912 may be substantially the same as the gas sensing probes 210, 212, 310 and 312 described with respect to FIGS. 2 and 3.

The electronic chip 904 may also have a circular or disc shape, as shown herein, or any other suitable shape. The electronic chip 904 may be configured to function substantially the same as the electronic chip 104 described with respect to FIG. 1. The electronic chip 904 may include an electronic circuit 934 having substantially the same configuration as the electronic chip 104 described with respect to FIG. 1. For example, the electronic circuit 934 of the electronic chip 904 may be formed on a flexible substrate 936. The electronic chip 904 may be coupled to the gas sensing probes 910 and 912 by electrical interconnects 926. The electronic circuit 934 may include a wireless transmitter configured to transmit a wireless signal indicating detection of the gas by the gas sensing probes 910 and 912.

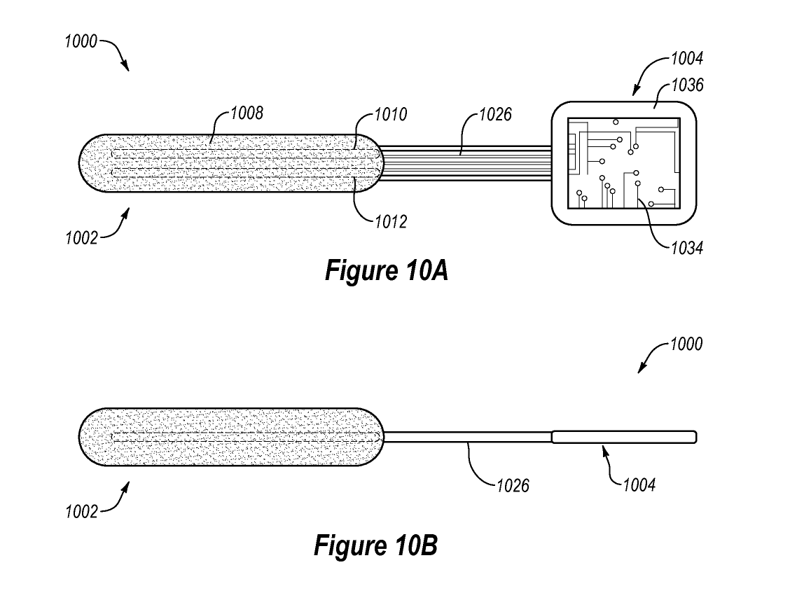

FIGS. 10A and 10B illustrate another embodiment of an apparatus 1000 for detecting gases in a body cavity, arranged in accordance with at least some embodiments described herein. FIG. 10A illustrates a top down view of the apparatus 1000 and FIG. 10B illustrates a side view of the apparatus 1000. The apparatus 1000 may include a probe 1002 including gas sensing probes 1010 and 1012 having a rod-shape positioned within a sheath 1008 having an elongated cylindrical shape. The gas sensing probes 1010 and 1012 may include one or more sensors configured to detect the presence of a gas, and may be coupled to an electronic chip 1004. For example, the gas sensing probes 1010 and 1012 may be substantially the same as the gas sensing probes 110, 112, 210, 212, 310 and 312 described with respect to FIGS. 2 and 3. The sheath 1008 may be formed from a biocompatible, semipermeable or permeable, rigid or semi-rigid material as described with respect to the sheath 108 of FIG. 1.

The electronic chip 1004 may also have a rectangular or square shape, as shown herein, or any other suitable shape. The electronic chip 1004 may include an electronic circuit 1034 having substantially the same configuration as the electronic chip 104 described with respect to FIG. 1. For example, the electronic circuit 1034 of the electronic chip 1004 may be formed on a flexible substrate 1036. The electronic chip 1004 may be coupled to the gas sensing probes 1010 and 1012 by electrical interconnects 1026. The electronic circuit 1034 may include a wireless transmitter configured to transmit a wireless signal indicating detection of the gas by the gas sensing probes 1010 and 1012.

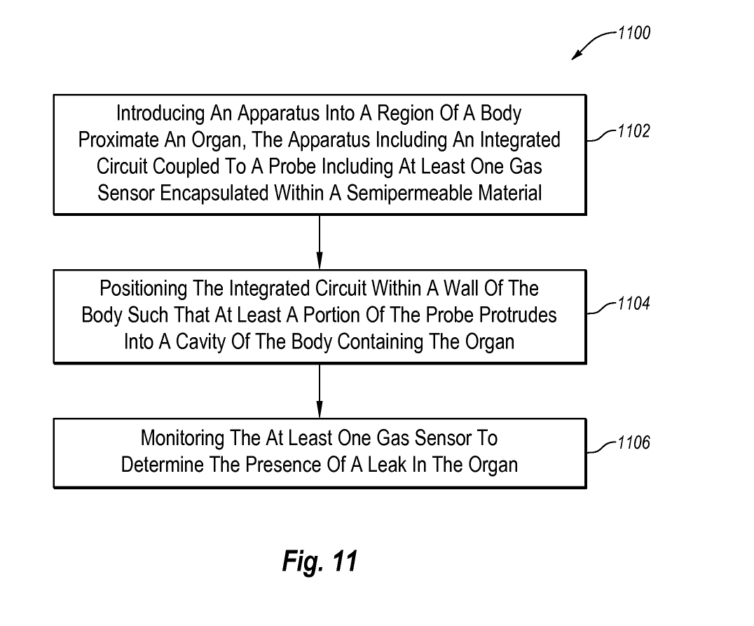

FIG. 11 shows an example flow diagram of a method for detecting leakage from a colon using the apparatus of FIGS. 1 through 6, arranged in accordance with at least some embodiments described herein. The method 1100 may be used for detecting leakage from organs, such as in detecting an anastomotic leak resulting from surgical connection of severed ends of at least one intestinal organ, such as the colon.

The method 1100 may be performed in whole or in part by using the apparatus 100, and optionally in conjunction with an external device, such as the external device 170 described with respect to FIG. 1. The method 1100 may include various operations, functions or actions as illustrated by one or more of blocks 1102, 1104, and/or 1106. The method 1100 may begin at block 1102.

In block 1102, [“Introducing An Apparatus Into A Region Of A Body Proximate An Organ, The Apparatus Including An Electronic Circuit Coupled To A Probe Including At Least One Gas Sensor Encapsulated Within A Semipermeable Material”], the method may begin with introducing an apparatus into a region of a body proximate an organ, the apparatus including an electronic circuit coupled to a probe including at least one gas sensor positioned within a semipermeable membrane. For example, the apparatus may be any one of the medical apparatus 100, 500, 600, 700, 800, 900 and 1000 of FIGS. 1 through 10B. Block 1102 may be followed by block 1104.

In block 1104, [“Positioning The Electronic Circuit Within A Wall Of The Body Such That At Least A Portion Of The Probe Protrudes Into A Cavity Of The Body Containing The Organ”], which may follow block 1102, the electronic circuit may be positioned within a wall of the body such that at least a portion of the probe protrudes into the cavity of the body containing the organ. The electronic circuit may be part of an electronic chip configured to receive information from the gas sensor and to transmit information related to detection of gases via wireless transmission, such as the electronic chip 104 of FIG. 1. Block 1104 may be followed by block 1106.

In block 1106, [“Monitoring The At Least One Gas Sensor To Determine The Presence Of A Leak In The Organ”], which may follow block 1104, that least one gas sensor may be monitored to determine the presence of a leak in the organ. For example, the gas sensor may be monitored using the external device 170 of FIG. 1.

The apparatus and methods described herein may, thus, enable direct detection of gases from an organ in the body as they occur, or shortly after. The apparatus may be introduced into the body of a patient during a surgical procedure and, as configured, conforms to the patient's anatomy such that it may remain in the body without harming surrounding tissues, causing the patient discomfort and/or hindering the patient from performing day-to-day activities. In addition, the medical apparatus may be introduced into the body of the patient while the patient is anesthetized and can remain in the peritoneal cavity for long periods of time. By enabling detection of aberrant gases inside the body, the apparatus may facilitate detection of leakage from organs in the body long before illness and/or symptoms cause by the leakage occur. Furthermore, the gases may be detected from small, unapparent lesions in the organ that may be overlooked using available testing methods. The gas sensors of the apparatus may be tailored to detect gases specific to a particular organ such that the apparatus may be useful in monitoring leakage that may result from surgery involving any organ in the body. The apparatus may be formed from component parts that are relatively inexpensive, biodegradable and lightweight.

The present disclosure is not to be limited in terms of the particular embodiments described herein, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into sub ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. An implantable apparatus for detecting leakage from an internal organ into a body cavity of a subject, comprising:
at least one gas sensing probe at least partially positioned within a semipermeable material and configured to detect an intestinal gas; and
an electronic circuit coupled to the at least one gas sensing probe and including a transmitter configured to transmit information from the at least one gas sensing probe to an external device located outside the subject,
wherein the at least one gas sensing probe and electronic circuit are configured for implantation into the subject.

2. The apparatus of claim 1, wherein the at least one gas sensing probe is configured to detect at least one of methane gas and hydrogen sulfide gas.

3. The apparatus of claim 1, wherein the at least one gas sensing probe includes at least one sensor having at least one material reactive with the intestinal gas.

4. The apparatus of claim 3, wherein the at least one sensor includes at least one of a metal oxide semiconductor device, a conducting polymer, a quartz crystal resonator, and a surface acoustic wave device.

5. The apparatus of claim 1, wherein the semipermeable material includes an internal passageway and the at least one gas sensing probe is surrounded by an activated carbon material.

6. The apparatus of claim 1, wherein the at least one gas sensing probe includes a first gas sensing probe configured to detect the presence of methane gas and a second gas sensing probe configured to detect the presence of hydrogen sulfide gas.

7. (canceled)

8. A system for detecting leakage from a colon into a body cavity of a subject, comprising:
an implant comprising:
at least one gas sensor positioned within a semipermeable sheath and configured to detect at least one of methane gas and hydrogen sulfide gas, the at least one gas sensor sized and configured to circumscribe at least a portion of the colon; and
an electronic circuit coupled to the at least one gas sensor and including an electronic transmitter, wherein the at least one gas sensor and electronic circuit are configured for implantation into the subject; and
an integrated receptor configured to receive a signal from the electronic transmitter of the electronic circuit.

9. The system of claim 8, wherein the at least one gas sensor includes an elongated sensor array positioned within an internal passageway of the semipermeable sheath.

10. The system of claim 8, wherein the semipermeable sheath is formed from a material impervious to liquids.

11. The system of claim 8, further comprising a filter material positioned between the at least one gas sensor and the semipermeable sheath, the filter material tailored to absorb the at least one of the methane gas and the hydrogen sulfide gas.

12. The system of claim 8, wherein at least one gas sensor includes an array of chemical sensors, each comprising a metal-oxide-semiconductor device configured to detect the methane gas or the hydrogen sulfide gas.

13. The system of claim 12, wherein each of the chemical sensors is configured to interact with the methane gas or the hydrogen sulfide gas to enable a change in conductivity in the metal-oxide-semiconductor device.

14. The system of claim 13, wherein the change in conductivity in the metal-oxide-semiconductor device activates the electronic transmitter of the electronic circuit to transmit an alert message to the integrated receptor.

15. The system of claim 8, wherein the electronic circuit is configured to use at least one of body heat and body movement as a power source.

16. The system of claim 8, wherein the electronic circuit includes at least one thermoelectric generator.

17. (canceled)

18. A method for detecting leakage from an internal organ into a body cavity of a subject, comprising:
introducing an implantable apparatus into an internal region of a body proximate an internal organ of the subject, the apparatus including an electronic circuit coupled to a probe including at least one gas sensor encapsulated within a semipermeable material;
positioning the electronic circuit within a wall of the body such that at least a portion of the probe protrudes into a cavity of the body containing the internal organ; and
monitoring the at least one gas sensor to determine the presence of gases leaked from the internal organ into the body cavity.

19. The method of claim 18, wherein introducing the implantable apparatus into the internal region of the body proximate the internal organ comprises introducing the apparatus into a preexisting opening in the body having dimensions of less than about 10 mm.

20. The method of claim 18, further comprising positioning the probe around at least a portion of the internal organ proximate an anastomosis.

21. The method of claim 18, further comprising fixing the electronic circuit to tissue of the wall of the body.

22. The method of claim 18, wherein monitoring the at least one gas sensor comprises one or both of:
monitoring detection of at least one of methane gas and hydrogen sulfide gas by the at least one gas sensor; or
detecting a change in conductivity of the gas sensor.

23. (canceled)

24. (canceled)
